(19) United States
(12) Patent Application Publication
(10) Pub. No.: US 2022/0031803 A1
(43) Pub. Date: Feb. 3, 2022

(54) ANTIBACTERIAL COMPOSITION COMPRISING MICROCIN S AND ANTIBIOTIC

(71) Applicant: SymbioGruppe GmbH & Co. KG, Herborn (DE)

(72) Inventors: Kurt ZIMMERMANN, Herbron (DE); Christian AUERBACH, Dresden (DE); Florian GUNZER, Dresden (DE)

(21) Appl. No.: 17/389,064

(22) Filed: Jul. 29, 2021

Related U.S. Application Data
(63) Continuation of application No. 15/989,790, filed on May 25, 2018.

(30) Foreign Application Priority Data
Nov. 29, 2017 (DE) 202017107259

(51) Int. Cl. A61K 38/16; A61P 31/04; A61P 1/00; A61K 31/7036; A61P 31/02; A61K 45/00; A61K 45/06; A61K 31/7048; C07K 14/245

(52) U.S. Cl. CPC A61K 38/164; A61P 31/04; A61P 1/00; A61K 31/7036; A61P 31/02; A61K 9/06; A61K 45/06; A61K 31/7048; C07K 14/245; Y02A 50/30; A61K 45/00

(57) ABSTRACT

A composition comprising an antimicrobial polypeptide and at least one antibiotic.

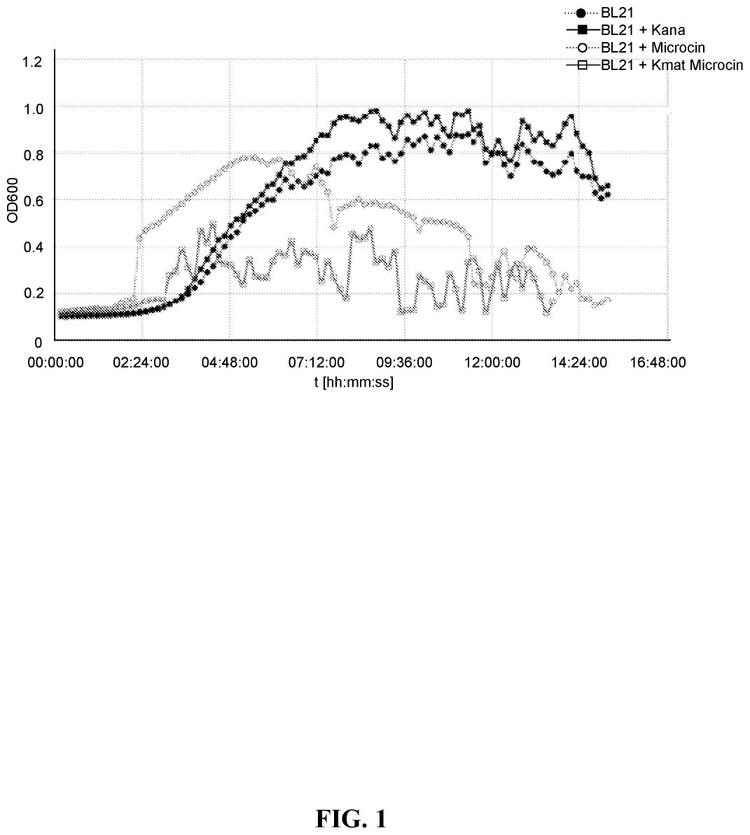

### CROSS REFERENCE TO RELATED APPLICATIONS

This application is a continuation of U.S. application Ser. No. 15/989,790, filed May 25, 2018, which claims priority to German Patent Application No. 202017107259, filed Nov. 29, 2017, the disclosure of each of which is hereby incorporated by reference in its entirety.

### BACKGROUND

The treatment of bacterial infections is largely based on the use of antibiotics. However, the widespread use of antibiotics has led to the emergence of multi-resistant bacteria which no longer respond to customary antibiotics. Therefore, there have recently been increased searches for alternatives to customary antibiotics. Alternatives that were contemplated included antimicrobial peptides produced by bacteria themselves. It was possible to demonstrate that microcins produced by bacteria can develop a strongly antimicrobial/antibacterial action against other bacteria that are closely related. An example of said microcins is microcin S, which is disclosed by WO 2013/024066.

Nevertheless, it would be desirable to provide further compositions which have an antimicrobial/antibacterial action.

### SUMMARY

It is therefore an object of the invention to provide compositions which have an antimicrobial/antibacterial action. It was found that, surprisingly, the combination of antibiotics and polypeptides having an antimicrobial action has a synergistic antimicrobial/antibacterial action.

Therefore, the object is achieved by a composition comprising an antimicrobial polypeptide and at least one antibiotic.

The antimicrobial polypeptide can comprise an amino acid sequence which is at least 70% identical to the amino acid sequence SEQ ID NO: 6 or SEQ ID NO: 7.

The antimicrobial polypeptide can be a naturally occurring allele variant of a polypeptide having the amino acid sequence SEQ ID NO: 6 or SEQ ID NO: 7. The antimicrobial polypeptide can be microcin S.

The antibiotic can be selected from the antibiotics of the group consisting of 3-lactams, glycopeptides, lipopeptides, polyketides, aminoglycoside antibiotics, polypeptide antibiotics, quinolones, sulfonamides, or a mixture thereof.

The antibiotic can be selected from the group consisting of amikacin, gentamicin, kanamycin, neomycin, netilmicin, streptomycin, tobramycin, or a mixture thereof.

The antibiotic can be kanamycin.

The composition can be that in which the composition comprises a quantity of microcin S and antibiotic that is suitable for achieving a synergistic antimicrobial effect.

The composition can be present as a pharmaceutical composition.

The pharmaceutical composition can comprise pharmaceutically tolerable excipients.

The pharmaceutical composition can be present as tablet, sugar-coated tablet, liquid, powder, or ointment.

Also disclosed is the use of the composition or the pharmaceutical composition for the treatment or prophylaxis of bacterial infectious diseases.

The bacterial infectious diseases can be caused by enteropathogenic and/or enterohaemorrhagic Escherichia coli or be associated with haemolytic uraemic syndrome.

Also disclosed is the use of the composition or the pharmaceutical composition for the treatment or prophylaxis of gastrointestinal disorders or diseases.

### BRIEF DESCRIPTION OF THE FIGURE

The FIGURE shows the growth curve of E. coli BL21(DE3)+kan^{R}. The bacteria were incubated without active ingredients (●), incubated with kanamycin (▪), incubated with microcin S (0), or incubated with kanamycin and microcin S (□). Growth was determined by measurement of the optical density at 600 nm (OD₆₀₀) for 18 hours in 96-well plates at 37° C. The profile of the OD₆₀₀ curves corresponds to the bacterial growth. Antibacterial activity is observed after 5 hours of incubation. Adding microcin S leads to a collapse in the bacterial growth after 5 hours. Additionally adding kanamycin prevents a strong bacterial growth in the initial hours of the observation period, and so the antibacterial effect is already observable after 3 to 3.5 hours. Each sample was prepared in duplicate and the results averaged.

### BRIEF DESCRIPTION OF THE SEQUENCE LISTING

SEQ ID NO: 1 is the nucleotide sequence of the microcin S operon of E. coli G3/10.

SEQ ID NO: 2 is the nucleotide sequence of the gene mcsS of E. coli G3/10.

SEQ ID NO: 3 is the nucleotide sequence of the gene mcsl of E. coli G3/10.

SEQ ID NO: 4 is the nucleotide sequence of the gene mcsA of E. coli G3/10.

SEQ ID NO: 5 is the nucleotide sequence of the gene mcsB of E. coli G3/10.

SEQ ID NO: 6 is the amino acid sequence of the microcin S precursor polypeptide of E. coli G3/10, including a leader sequence.

SEQ ID NO: 7 is the amino acid sequence of the microcin S polypeptide of E. coli G3/10 without the leader peptide.

SEQ ID NOs: 8-26 are oligonucleotide primers used to amplify genes present in the microcin S operon (SEQ ID NO: 1), or probes for screening for the mcsS gene coding for the microcin S polypeptide.

### DETAILED DESCRIPTION

### Definitions

Unless defined otherwise, all the technical and scientific terms used here have the same meaning, as customarily understood by a person skilled in the art in the field of the invention. In the event of a conflict, the definition specified in this description takes precedence.

The articles “a”, “an” or “the” refer to “one” or “more” whenever used here, i.e. what is meant is “one”, “at least one” or “one or more”. For example, the term “an antibiotic” can refer both to an individual antibiotic and to a multiplicity of antibiotics.

The term “comprise” also encompasses embodiments in which the term “comprises” means “consists of”.

As used here, the identity of the amino acid or of the nucleic acid molecule is equivalent to the “homology” of the amino acid or of the nucleic acid molecule. The expression “stringent conditions” refers to conditions under which a probe nucleic acid molecule will hybridize to its target nucleic acid molecule sequence, typically in a complex mixture of nucleic acid molecules, but not to any other sequences. Stringent conditions are sequence-dependent and differ according to the circumstances.

Longer sequences hybridize in a specific manner at higher temperatures.

Nucleic acid hybridization parameters can be found in the relevant literature, which provide such methods, for example Molecular Cloning: A Laboratory Manual, J. Sambrook et al., editors, second edition, Cold Spring Harbor Laboratory Press, Cold Spring Harbour, New York, 1989, or aktuelle Protokolle in der Molekularbiologie [Current protocols in molecular biology], FM Ausubel et al., editors, John Wiley & Sons, Inc., New York. In general, stringent conditions are selected such that they are about 5-10° C. below the thermal melting point (Tₘ) for the specific sequence at a defined ionic strength-pH. The Tₘ is the temperature (under defined ionic strength, pH and nucleic acid concentration) at which 50% of the probes complementary to the target hybridize at equilibrium to the target sequence (since the target sequences are present in excess at the Tₘ, 50% of the probes are hybridized at equilibrium).

Stringent conditions are those at which the salt concentration is less than about 1.0 M sodium ions, typically about 0.01 to 1.0 M sodium ions (or other salts), at pH 7.0 to 8.3 and the temperature is at least about 30° C. for short probes (e.g. 10 to 50 nucleotides) and at least about 60° C. for long probes (e.g. more than 50 nucleotides). Stringent conditions can also be achieved through the addition of destabilization agents such as formamide. For hybridization with high stringency, a positive signal is at least twice the background, preferably 10 times the background hybridization. Exemplary highly-stringent or stringent hybridization conditions comprise: 50% formamide, 5×SSC and 1% SDS, incubated at 42° C., or 5×SSC and 1% SDS, incubated at 65° C., with washes in 0.2×SSC and 0.1% SDS at 65° C.

As used herein, the term “microcin-like activity” of a polypeptide means that the polypeptide exerts a strong antimicrobial/antibacterial activity against closely related species. It additionally means that microcin producers are resistant against the microcin which they have produced, which is mediated by at least one resistance-imparting gene within a gene cluster.

As used here, the term “plasmid” refers to a nucleic acid molecule which is capable of replication in a cell and can be functionally bonded to another nucleic acid molecule in order to bring about the replication of the attached segment. Plasmids capable of controlling the expression of a structural gene coding for a subject polypeptide are referred to herein as “expression plasm ids”.

As used herein, the expression “functionally linked” means that the available nucleic acid molecule is bonded to the plasmid, with the result that the expression of the structural gene formed by the nucleic acid molecule is under the control of the plasmid. The term “regulatory sequence” is intended to include promoters, enhancers and other expression control elements (e.g. polyadenylation signals).

Herein, the term “conjugative plasmid” refers to a plasmid which can move from one cell to another during the conjugation process.

As used here, the terms “transformation” and “transfection” are intended to refer to a multiplicity of techniques for introducing foreign nucleic acid (e.g. DNA) into a host cell, which techniques are recognized in the technical field and include calcium phosphate or calcium chloride coprecipitation, DEAE-dextran-mediated transfection, lipofection or electroporation.

As used herein, a “pharmaceutical composition” refers to a preparation of one or more of the active ingredients described herein, i.e. the polypeptide according to the invention (or a pharmaceutically tolerable salt thereof) and an antibiotic according to the invention, with other chemical components such as physiologically and pharmaceutically tolerable carriers and excipients. The purpose of a pharmaceutical composition is that of facilitating the administration of a compound to an organism.

Hereinbelow, the expressions “physiologically tolerable carrier” and “pharmaceutically tolerable carrier”, which can be used interchangeably, refer to a carrier or a diluent which does not cause significant irritation of an organism and does not cancel out the biological activity and properties of the administered active ingredients. One of the constituents present in the pharmaceutically acceptable carrier can, for example, be polyethylene glycol (PEG), a biocompatible polymer having a wide solubility range both in organic and in aqueous media.

Herein, the expression “excipient” refers to an inert substance which is added to a medicament in order to further facilitate the administration of an active ingredient. Examples of excipients comprise calcium carbonate, calcium phosphate, various sugars and starch types, cellulose derivatives, gelatin, vegetable oils and polyethylene glycols.

As used herein, the term “treatment” refers to the prevention, curing, reversing, weakening, alleviation, minimization, suppression or stopping of the harmful effects of a disease process. As used here, the term “subject” refers to a subject which may benefit from the present invention, such as an animal or mammal (e.g. dog, cat, sheep, pig, horse, cattle, human), preferably a human subject.

Herein, the term “functional gastrointestinal disorder” encompasses a number of separate idiopathic disorders which affect different parts of the gastrointestinal tract.

To determine the percentage identity of two amino acid sequences or of two nucleic acid sequences, the sequences are aligned for optimal comparative purposes (e.g. it possible to insert gaps into one or both of a first and a second amino acid or nucleic acid sequence for optimal alignment).

The residues at corresponding positions are then compared, and if a position in one sequence is occupied by the same residue like the corresponding position in the other sequence, then the molecules are identical at this position. The percentage identity between two sequences is therefore a function of the number of identical positions shared by two sequences (i.e. % identity=#identical positions/total number of positions×100). The percentage identity between the two sequences is a function of the number of identical positions jointly used by the sequences, wherein the number of gaps and the length of each gap are taken into account, which gaps are inserted for optimal alignment of the two sequences.

### Composition

A composition comprising a polypeptide having an antimicrobial action, more particularly microcin S, and at least one antibiotic is disclosed.

### Antimicrobial Polypeptides

The composition comprises an antimicrobial polypeptide, wherein the polypeptide a) comprises an amino acid sequence which is at least 50%, 60%, 70%, 80%, 90%, 95% or more identical to the amino acid sequence of SEQ ID NO: 6 or of SEQ ID NO: 7;

b) is coded by a nucleic acid molecule comprising a nucleotide sequence which is at least 50%, 60%, 70%, 80%, 90%, 95% or more identical to a nucleotide sequence comprising the nucleotide sequence of one of SEQ ID NOs: 1, 2, 3, 4 or 5 or a complement thereof;

c) is coded by a nucleic acid molecule which hybridizes to a nucleotide sequence complementary to a nucleotide sequence comprising the sequence of nucleotides of one of SEQ ID NOs: 1, 2, 3, 4 or 5 or a complement thereof under stringent conditions; or
d) comprises a naturally occurring allele variant of a polypeptide comprising the amino acid sequence of SEQ ID NO: 6 or of SEQ ID NO: 7.

The term “antimicrobial activity” is to be understood to mean antimicrobial activity caused by microcin, more particularly by microcin S.

In a further aspect of the invention, an isolated nucleic acid molecule encoding a microcin S polypeptide is provided, wherein the nucleic acid molecule

a) comprises a nucleotide sequence which is at least 50%, 60%, 70%, 80%, 90%, 95% or more identical to one of SEQ ID NOs: 1, 2, 3, 4 or 5 or a complement thereof;
b) comprises a nucleic acid molecule which hybridizes to a nucleotide sequence complementary to a nucleotide sequence comprising the nucleotide sequence of one of SEQ ID NOs: 1, 2, 3, 4 or 5 or a complement thereof under stringent conditions;
c) comprises a nucleotide sequence encoding a polypeptide comprising an amino acid sequence which is at least 50%, 60%, 70%, 80%, 90%, 95% or more identical to the amino acid sequence of SEQ ID NO: 6 or of SEQ ID NO: 7; or
d) comprises a nucleic acid molecule coding for a naturally occurring allele variant of a polypeptide comprising the amino acid sequence of SEQ ID NO: 6 or of SEQ ID NO: 7.

The composition comprises a polypeptide as described above in a quantity of microcin S and antibiotic that is suitable for achieving a synergistic antimicrobial effect. The synergistic effect can, for example, be determined by means of the test method described in the example.

### Antibiotic

The composition contains at least one antibiotic. The antibiotic is not one of the above-described antimicrobial polypeptides.

The antibiotic is selected from the antibiotics of the group consisting of 3-lactams, glycopeptides, lipopeptides, polyketides, aminoglycoside antibiotics, polypeptide antibiotics, quinolones, sulfonamides, or a mixture thereof.

In particular, the composition can contain 1, 2, 3, 4, 5, 6, or 7 different antibiotics of the above-specified group.

Preferably, the antibiotic is selected from the group of aminoglycoside antibiotics consisting of amikacin, gentamicin, kanamycin, neomycin, netilmicin, streptomycin, tobramycin, or a mixture thereof.

The antibiotic can be selected from the group of β-lactams or a mixture of β-lactams consisting of
penicillins, comprising
   β-lactamase-sensitive (-unstable) penicillins (e.g. benzylpenicillin (penicillin G), phenoxymethylpenicillin (penicillin V), propicillin, azidocillin),
   β-lactamase-resistant (-stable) penicillins (e.g. flucloxacillin, dicloxacillin, cloxacillin, oxacillin, methicillin, broad-spectrum penicillins)
   broad-spectrum penicillins
      aminopenicillins (e.g. amoxicillin, ampicillin, bacampicillin),
      acylaminopenicillins (e.g. mezlocillin, piperacillin, pivmecillinam),
cephalosporins, comprising
   cephalosporins for parenteral use
      cephalosporins without increased β-lactamase stability (base cephalosporins)
      cephalosporins with increased β-lactamase stability (e.g. cefuroxime, cefamandole, cefoxitin, cefotiam)
      road-spectrum cephalosporins (e.g. cefotaxime, cefovecin, ceftazidime, cefepime, cefodizime, ceftriaxone)
oral cephalosporines
   oral cephalosporines without increased β-lactamase stability (e.g. cefaclor, cefadroxil, cefalexin, loracarbef)
   oral cephalosporines with increased β-lactamase stability (e.g. cefixime, cefuroxime axetil, cefetamet pivoxil, ceftibuten, cefpodoxime proxetil)
β-lactamase inhibitors (e.g. clavulanic acid in combination with amoxicillin, sulbactam, tazobactam in combination with piperacillin),
uncategorized β-lactam antibiotics
carbapenems (e.g. imipenem in combination with cilastatin, meropenem, doripenem, ertapenem),
monobactams (aztreonam).

The antibiotic can be selected from the group of glycopeptides consisting of vancomycin, dalbavancin, teicoplanin, or a mixture thereof.

The antibiotic can be selected from the group of lipopeptides consisting of daptomycin.

The antibiotic can be selected from the group of polyketides consisting of tetracyclines or macrolide antibiotics (e.g. lincosam ides or/and oxazolidinones)

The antibiotic can be selected from the group of quinolones consisting of cinoxacin, ciprofloxacin, clioquinol, danofloxacin, difloxacin, enrofloxacin, fleroxacin, flumequine, gatifloxacin, grepafloxacin, ibafloxacin, levofloxacin, marbofloxacin, moxifloxacin, nalidixic acid, norfloxacin, ofloxacin, orbifloxacin, oxolinic acid, pipemidic acid (pyridopyrimidine), sarafloxacin, sparfloxacin, temafloxacin, or nadifloxacin.

The antibiotic can be selected from the group of sulfonamides consisting of sulfacarbamide, mafenide, sulfaguanidine, sulfacetamide, sulfathiazole, sulfamethizole, sulfametrole, sulfamethylthiazole, sulfachloropyridazine, sulfachloropyrazine, sulfadiazine, sulfamethoxazole, sulfapyridine, sulfamerazine, sulfaperin, sulfamethoxypyridazine, sulfamethoxydiazine, sulfalene, sulfamoxole, sulfafurazole, sulfadicramide, sulfadimidine, sulfisomidine, sulfametomidine, sulfadimethoxine, sulfadoxine, sulfaphenazole, or sulfasalazine.

### Pharmaceutical Compositions

In a preferred embodiment, the composition can be formulated as a pharmaceutical composition. Then, the pharmaceutical composition further comprises a pharmaceutically tolerable carrier. The pharmaceutical composition can contain a therapeutically effective quantity of the active ingredients and one or more adjuvants, excipients, carriers and/or diluents. Physiologically tolerable diluents, carriers and excipients typically influence the homeostasis of a recipient (e.g. electrolyte balance) non-disadvantageously. Acceptable carriers include biocompatible, inert or bioabsorbable salts, buffering agents, oligo- or polysaccharides, polymers, viscosity-improving agents, preservatives and the like. Further details on techniques for formulating and administering pharmaceutical compositions can be found, for example, in REMINGTON'S PHARMACEUTICAL SCIENCES (Maack Publishing Co., Easton, Pa.).

Examples of additives comprise glucose, lactose, sucrose, mannitol, starch, cellulose or cellulose derivatives, magnesium stearate, stearic acid, sodium saccharin, talc, magnesium carbonate and the like. Examples of additives which can be added in order to provide a desirable colour, a desired taste, a desired stability, buffering capacity, dispersion or other known desirable features are red iron oxide, silica gel, sodium lauryl sulfate, titanium dioxide, edible white ink and the like. Similar diluents can be used in order to produce compressed tablets.

In certain embodiments, it is also possible to incorporate additional active ingredients into the composition.

The pharmaceutical compositions of the invention are formulated such that they are compatible with their intended route of administration. Examples of routes of administration comprise parenteral (e.g. intravenous, intradermal, subcutaneous), oral, transdermal, topical, transmucosal and rectal administration.

Solutions or suspensions used for parenteral, intradermal or subcutaneous application can comprise the following components: a sterile diluent such as water for injection, saline solution, fixed oils, polyethylene glycols, glycerol, propylene glycol or other synthetic solvents; antibacterial agents such as benzyl alcohol or methylparaben; antioxidants such as ascorbic acid or sodium bisulfite; chelating agents such as ethylenediaminetetraacetic acid; buffers such as acetates, citrates or phosphates and agents for adjusting tonicity such as sodium chloride or dextrose. The pH can be adjusted using acids or bases, such as hydrochloric acid or sodium hydroxide. The parenteral preparation can be enclosed in vials, disposable syringes or multi-dose bottles made of glass or plastic.

Compositions suitable for an injectable use encompass sterile aqueous solutions or dispersions and sterile powders for the preparation of sterile injectable solutions or dispersions. Carriers suitable for intravenous administration encompass physiological saline solution, bacteriostatic water, Cremophor EL TM (BASF, Parsippany, N.J.) or phosphate-buffered saline solution (PBS). In all cases, the composition must be sterile and should be sufficiently liquid for there to be an easy injectability. The composition must be stable under the preparation and storage conditions and must be protected from the contaminating action of microorganisms such as bacteria and fungi. The carrier can be a solvent or a dispersion medium which contains, for example, water, ethanol, polyol (e.g. glycerol, propylene glycol and liquid polyethylene glycol and the like) and suitable mixtures thereof. The right fluidity can, for example, be maintained by the use of a coating such as lecithin, by the maintenance of the required particle size in the case of the dispersion and by the use of surface-active agents.

Preventing the action of microorganisms can be achieved by various antibacterial and antimycotic agents, for example parabens, chlorobutanol, phenol, ascorbic acid, thimerosal and the like. In many cases, it is preferred to incorporate isotonic agents in the composition, for example sugars, polyalcohols, such as mannitol and sorbitol, sodium chloride. An extended absorption of the injectable compositions can be achieved by incorporating in the composition an agent which delays absorption, for example aluminium monostearate and gelatin.

Oral administration can be achieved in the form of a capsule, a liquid, a tablet, a pill or a formulation with extended release.

Oral compositions generally comprise an inert diluent or an edible carrier. They can be incorporated in gelatin capsules or compressed to form tablets. For the purposes of oral therapy when administering, the active compound can be introduced with excipients and used in the form of tablets, lozenges or capsules. Oral compositions can also be prepared using a fluid carrier, wherein the compound in the fluid carrier is orally applied and rinsed and coughed up or swallowed. Pharmaceutically compatible binders and/or adjuvant materials can be present as part of the composition. The oral composition can contain one of the following constituents or compounds of a similar nature: a salt such as sodium chloride, magnesium sulfate, such as magnesium sulfate.7H₂O, potassium chloride, calcium chloride, such as calcium chloride. 2H₂O, magnesium chloride, such as magnesium chloride.6H₂O; purified water; a binder such as microcrystalline cellulose, tragacanth or gelatin; an excipient such as starch or lactose; a disintegrant such as alginic acid, primogel or corn starch; a lubricant such as magnesium stearate or Sterotex; a glidant such as colloidal silicon dioxide; a sweetener such as sucrose or saccharin; or a flavouring such as peppermint, methyl salicylate or orange flavouring.

For administration by inhalation, the compounds are delivered in the form of an aerosol spray from a pressurized container or dispenser which contains a suitable propellant, for example a gas such as carbon dioxide, or an atomizer.

Systemic administration can also be achieved transmucosally or transdermally. For transmucosal or transdermal administration, penetrants suitable for the barrier to be penetrated are used in the formulation. Such penetrants are known in general in the prior art and encompass, for example for transmucosal administration, detergents, bile salts and fusidic acid derivatives.

Transmucosal administration can be achieved by the use of nasal sprays or suppositories. For transdermal administration, the active compounds are formulated to form ointments, gels or creams, as is known in general in the technical field.

The compounds can also be prepared in the form of suppositories (e.g. with conventional suppository bases, such as cocoa butter and other glycerides) or retention enemas for rectal delivery.

In one embodiment, the active compounds are prepared with carriers which protect the compound from a rapid elimination from the body, such as a formulation with controlled release, including implants and microencapsulated delivery systems. Biologically degradable, biocompatible polymers can be used, such as ethylene vinyl acetate, polyanhydrides, polyglycolic acid, collagen, polyorthoester and polylactic acid. Methods for preparing such formulations are obvious to a person skilled in the art. The materials can also be obtained commercially from Alza Corporation and Nova Pharmaceuticals, Inc. Liposomal suspensions (including liposomes directed to infected cells with monoclonal antibodies against viral antigens) can likewise be used as pharmaceutically acceptable carriers. They can be prepared in accordance with methods known to those skilled in the art, for example as described in U.S. Pat. No. 4,522,811.

It is particularly advantageous to formulate oral compositions in a dosage-unit form to facilitate the administration and uniformity of the dosage. The dosage-unit form, as used here, refers to physically discrete units suitable as uniform dosages for the subject to be treated; each unit contains a predetermined quantity of active compounds that is calculated such that the desired therapeutic action is generated in conjunction with the required pharmaceutical carrier. The specifications for the dosage-unit forms of the invention are determined by the unique characteristics of the active compound and the particular therapeutic action to be achieved and the restrictions inherent, in the technical field of the compound, to such an active compound for the treatment of individuals.

### Kit

Kits comprising a therapeutically or prophylactically effective quantity of the composition of the invention in one or more containers are likewise provided. The kit can optionally comprise instructions for carrying out the intended medical uses, as disclosed herein, or for carrying out the provided methods. The kit comprises at least one of the above-described antimicrobial polypeptides and at least one of the above-described antibiotics. The polypeptide and the antibiotic can be present separated from one another.

### Medical Uses of the Compositions and of the Kit

Since the polypeptide according to the invention in combination with the at least one antibiotic exhibits a synergistic antimicrobial or antibacterial activity, the invention further provides for the medical uses of the composition or of the kit in therapy or prevention.

In particular, they can be used in the treatment or prevention of microbial infections. Furthermore, they are used in the treatment and prophylaxis of gastrointestinal disorders, for example functional gastrointestinal disorders, and, in one embodiment, in the treatment and prophylaxis of diarrhoea.

In a preferred embodiment, the microbial infection encompasses infections with enteropathogenic and/or enterohaemorrhagic E. coli (EPEC, EHEC). In a further embodiment, the microbial infection encompasses a haemolytic uraemic syndrome (HUS), preferably an enteropathic haemolytic uraemic syndrome. However, the invention is not restricted to diseases caused by EPEC or EHEC, but also encompasses the treatment or prophylaxis of diseases caused by other microorganisms, which for gastrointestinal disorders such as diarrhoea, for example Salmonella spp., Shigella spp. and Yersinia spp.

In a preferred embodiment, the microbial infection encompasses an enterohaemorrhagic E. coli (EHEC) infection. This means the enterohaemorrhagic E. coli O104: H4 outbreak strain or any other EHEC vulnerable to microcin S. In a further preferred embodiment, the microbial infection encompasses a Shiga toxin-producing E. coli infection. The Shiga toxin-producing E. coli can be an enterohaemorrhagic E. coli. Besides other virulence factors, Shiga toxins (Stx 1; Stx 2; Stx 1,2) are responsible for severe, usually bloody diarrhoea and the occurrence of haemolytic uraemic syndrome. EHEC cells release relatively high quantities of Shiga toxins as a result of treatment with certain antibiotics. Therefore, antibiotic treatment of patients with EHEC infections is contraindicated in the case of certain antibiotics.

By treating the microbial infection using the present composition containing antibiotics which do not release Shiga toxins, it is possible to inhibit the production of Shiga toxin.

In other embodiments, the microbial infection encompasses an enteropathogenic E. coli infection.

In some embodiments, the microbial infection encompasses a beta-lactamase-producing Enterobacteriaceae infection with extended spectrum. Preferably, the microbial infection encompasses a beta-lactamase-producing E. coli with extended spectrum.

It is preferred that they are used in the treatment of adults and children.

In some embodiments described here, the composition can be administered to a subject in parenteral (e.g. intravenous, intradermal, subcutaneous), oral, transdermal, topical, transmucosal or rectal form, preferably in oral form.

The composition can be administered to a subject in a pharmaceutically effective quantity. Administration of a pharmaceutically effective quantity of the compositions of the present invention is defined as an effective quantity at dosages and for time spans that are required in order to achieve the desired result. For example, a pharmaceutically effective quantity of a composition can vary depending on factors such as the disease state, age, gender and weight of the individual and the capacity of the polypeptide; the cell or the compositions for causing a desired response in the individual. The dosage regimen can be adapted in order to provide the optimal therapeutic response. For example, multiple divided doses can be administered daily or the dose can be reduced proportionally in line with the requirements of the therapeutic situation.

To optimize therapeutic efficacy, the composition is administered at different time points in different dosage schemes. The subject can be administered with an individual pharmaceutically effective dose or multiple pharmaceutically effective doses, for example 2, 3, 4, 5, 6, 7 or more. Specifically, the subject can be administered with an individual pharmaceutically effective dose 2, 3, 4, 5, 6, 7 or many times per day.

In particular, the subject can be administered with one dose of 5-15 drops of an aqueous composition. Preferably, one dose of 10 drops is to be administered. In one such embodiment, 1 ml contains about 14 drops.

A pharmaceutically effective quantity (i.e. a pharmaceutically effective dose) of the polypeptide in the composition is in the range from about 0.001 to 30 mg per kg of body weight, preferably about 0.01 to 25 mg per kg of body weight, more preferably about 0.1 to 20 mg per kg of body weight and yet more preferably about 1 to 10, 2 to 9, 3 to 8, 4 to 7 or 5 to 6 mg per kg of body weight.

The dose of the antibiotic is to correspond to the dose of the antibiotic administered to the subject if the antibiotic were to be administered as sole active ingredient without admixture of the polypeptide, this being referred to here as usual dose. The dose of the antibiotic can be 10%, 20%, 30%, 40%, 50%, 60%, 70%, 80%, 90%, 95%, or 99% below the usual dose when it is formulated in the composition according to the invention.

Administrations with multiple doses can be separated by intervals of hours, days, weeks or months. In further embodiments, they are administered at least once, twice or thrice daily with meals in water, preferably thrice daily, with meals in water.

The composition can, if necessary, be administered to the subject for a limited period and/or in a limited number of doses. For example, in some embodiments, the administration to the subject can be ended within, for example, one year, six months, one month or two weeks (i.e. no further administrations are provided). For example, the provided administration can be ended after six months. In the case of chronic diseases, it may be necessary to extend the period to up to six months.

In some embodiments, the dose can be increased after 2 days, 3 days, 4 days, 5 days, 6 days, 1 week, 2 weeks, 3 weeks, 4 weeks or more. The dose to be administered to a person can be increased to 15 to 25 drops, more preferably to 20 drops of an aqueous solution.

### EXAMPLES

### Example 1: Preparation of Microcin S

Microcin S was prepared in a cell-free E. coli based in vitro expression system over an 18 h incubation at 27° C. having the following components:
Maltodextrin (35 mM)
Glucose (60 mM)
Nucleotides (each 2.25 mM)
cAMP (1.13 mM)
NAD (0.5 mM)
Coenzyme A (0.38 mM)
DNA template (10 nM)
Cell extract from E. coli containing ribosomes (9.2 mg/ml), also contains T7 RNA polymerase
Glutathione/glutathione disulfide (6 nM)
Hepes (pH 8.0) (75 mM)
Amino acids (each 2 mM)
tRNA (0.3 mg/ml), Mg glutamate (12.5 mM), K glutamate (60 mM), folic acid (0.1 mM), spermidine (0.5 mM), PEG 8000 (3.5%).

Expression is initiated by addition of the T7 RNA polymerase. The template for the microcin S are circular DNA molecules, wherein the DNA molecules for the microcin S encoded a 6×histidine tag C-terminally.

### Example 2: Antimicrobial Activity of Microcin S and a Combination of Microcin S and Antibiotics

Antimicrobial activity was investigated in a 96-well plate by incubating E. coli BL21 (DE3)+kan^{R} bacteria either with microcin S alone or with microcin S and an antibiotic at 37° C. for 18 h. The controls used were incubations without addition of active ingredients and incubations with only antibiotic. The growth profile of the incubated bacteria was tracked by means of measurement of the optical density at 600 nm (OD 600) and the end points of the investigation after 18 h are reported in the following table:

| | | Microcin S + | No active | |
|---|---|---|---|---|
| | Microcin S | kanamycin | ingredients | Kanamycin |
| Bacterial | ++ | ++++ | − | − |
| agglutination | | | | |

To investigate whether microcin S in combination with antibiotics can likewise achieve synergistic effects on sensitive bacteria, growth profiles of the microcin S-sensitive indicator strain Escherichia coli BL21 (DE3) provided with a kanamycin resistance gene are recorded photometrically in a microtitre plate of 96-well format. At a total volume of 150 μl per well, the culture medium is inoculated to an OD600 of 0.1 with the indicator strain and, where appropriate, together with 50 μg/ml kanamycin and/or 10 μl of sterile microcin solution (supernatant of the cell-free expression reaction). At the same time, the wells containing only bacteria or bacteria and kanamycin served as growth control. Since it is also possible to investigate the sensitivity of a bacterial strain with respect to MccS using this method, the indicator strain is incubated together with MccS to monitor the antimicrobial action of MccS. To monitor sterility, no bacteria are added to the relevant wells. This simultaneously serves as blank for the photometer to reduce the background absorption. After loading with the individual samples, the microtitre plate is incubated directly in a photometer (Epoch2 microplate reader from Biotek, USA) at 37° C. and 200 rpm (double-orbital, ∞) for 15 h. The optical density at 600 nm wavelength (OD₆₀₀) is measured simultaneously for all wells at regular intervals. The graphic evaluation of the recorded measurement points is done subsequently with the aid of Microsoft Excel (Microsoft Corporation, USA). Differences in the growth behaviour between the various preparations are used to discuss possible synergistic effects between MccS and kanamycin.

It was possible to demonstrate that the antimicrobial effect of microcin S could be substantially enhanced by addition of an antibiotic. This is particularly remarkable because the antibiotic without addition of microcin S to the kanamycin-resistant E. coli strain exhibited, expectably, no detectable antimicrobial action on bacterial growth.

1. A composition comprising an antimicrobial polypeptide and at least one antibiotic, wherein the antimicrobial polypeptide is an amino acid sequence which is at least 90% identical to the amino acid sequence SEQ ID NO:6 or SEQ ID NO:7; or
microcin S, and wherein
the antibiotic is a polypeptide antibiotic.

2-7. (canceled)

8. The composition according to claim 1, characterized in that the composition comprises a quantity of microcin S and antibiotic that is suitable for achieving a synergistic antimicrobial effect.

9. A pharmaceutical composition comprising an antimicrobial polypeptide and at least one antibiotic, wherein the antimicrobial polypeptide is an amino acid sequence which is at least 90% identical to the amino acid sequence SEQ ID NO:6 or SEQ ID NO:7, or
microcin S, and wherein
the antibiotic is a polypeptide antibiotic.

10. The pharmaceutical composition according to claim 9, characterized in that the composition comprises pharmaceutically tolerable excipients.

11. The pharmaceutical composition according to claim 10, characterized in that it is present as tablet, sugar-coated tablet, liquid, powder, or ointment.

12. A method of treatment or prophylaxis of bacterial infectious diseases characterized in that a pharmaceutically effective amount of a composition according to claim 9 is administered to a subject in need thereof.

13. The method according to claim 12, characterized in that the bacterial infectious diseases are caused by enteropathogenic and/or enterohaemorrhagic Escherichia coli or are associated with haemolytic uraemic syndrome.

14. The method according to claim 12, characterized in that the treatment or prophylaxis of bacterial infectious diseases involves the treatment or prophylaxis of gastrointestinal disorders or diseases, septic symptoms, multi-resistant pathogens, or sepsis with multi-resistant pathogens.

15. The pharmaceutical composition according to claim 9, characterized in that it is present as tablet, sugar-coated tablet, liquid, powder, or ointment.


SEQUENCE LISTING (26 sequences)

SEQ ID NO: 1 - DNA, 4679 bp, Escherichia coli
ttaatttttgttcagcccatcatcaatggtgcgcttgtgcctcagctccataaagcagccgtctgccagatacagaacccggtctgcagaggctatggtttccgggcgatgggctatcagcaggacaggaagccccagctgacgtagtgtctggcttatctgaatttcactttcaacatcaagatggctggtcgcttcatccagtaataaaaatcccggtttcttgtacaaagccctggccagcagaatgcgttgtttttgccctccggaaagccctccaccggtttcaccaatcagcgtctgataccccatcggcatagacatgatatcgctgtcgataagtgccagtctggcgcactggatcattcgttcatgattgcggtctccactgaaaaaaatgatgttatctgcaatggagcccctgaaaagatggtcatcctgcagtacggtaccgattcgctgacgtacctgaaaataatccgggtgggtatgcggtatgccaaatgccctgattgttccctcatccgggatgtggatccccaggataagcttcaccagtgtcgatttcccgcatccggattgaccggtaatggccatcacctcgccaggatggagtgtcagcgacaccccgcggagtatcggtttattccctcccttgtggctgaaggttatatgctcaagggataaaggaacacgggcaccgtcggccacatccccccggaatacggacgggaagacagaagtatcattgtcttcctgagcgggttgttgctgatagtcttctgtcggggtcagcacaatgtcagccaggcgttcgttgtagatatcaagcatacgccaggaaaaatacttatcggtcaggctgctgatactggatgaaaaacgcatctggtaggacaaataagcaaccagcatgccgacggtgaaggtaccgttcagtacttccccggcacctttccacaaaataatcgctgataccacgctgccggtcagggtgtgtgcaatgtcatagcacatcagctgacggctctgtcgcagctgcgtattcctgcgaacaatgttgaggttcagccaggcggcctcacggtgcgctgtcacgccgttaattctcaggctcagaataccgttgagggtttcaagaaaatgcccggactccctggcacctgcgtcccaggcatcttctgccgactgtcgaagggacggataccacaacgccctcagcacaccgtagataatggctgccagtacggctatcagcgtcatttccggactgtacagcagcatcatacagagcgcagttatcacgagcaacacgtccagaataccttccagtacctgcgtggtcagcgcctcctggatggtatccacggcctcaaaacgggcattgatacttcccttactgcgcgcatcaaaccagcccagcgggagacgcaccagatgatggaaaacccgggctgtccactgcatgttaaaattgacggacagagttatactggcccactggcgtgccagcgacagcagcatctgggtcattgacagcagcagcagggcaatgattataacggtcaggagacttcgatcggcggcaaccagcacttcatcaattaccagctggttgagaagtggactactcagcgtcaggatttccagcgccagggcaaaaatgatgatgcgcgacatcgcggccagcagcccgggtgtcctgcctgtcagttggcgcaggcgaattttttttctttcatcccgggcggtaaaatcactggccggcgtcaactccagcgcgacaccggtgaaatgttttcctgcatcctgcagggagagcgttatcttccctttgtccgggtcgtggataaccagcctgctcccgcgaactttataaagcacgacgaaatggttcatgttccagtggagaatgcacgggagagaaagggatttcaggtcttccggctccagccggacggcccgggaggacaaccggataccggctgcacactctatcagcctctgcagggtcatcccctgtgtgctgatactgaaacgttcgcgcagggtgggtaaatctgtctgcagaccatgccagcaggccatcatgaccagacatgccagaccacattccgcagactctgtctgccggatgagggggagctgttttctcccggtccatttaagggtatccattacagttttcctttcaggctccagagtggctcggtcagccactcccacaaatggcgggtatccaggctgacatccccctcaagggtcatgccggggcgcaggggttcctgttttccataagccagaatgaaggtattctcaggttcgacgatgacgcggtagtgcccttcattctctttccaggtcacaggggatacggagagtaaatccgaaggggtcagcgttgtacggcttatctctcggatagtcccgtactgaacgccaaacttctgataggggaatgcggcgaaccgaagtgcaacacgctgccccggctgaataaaaccggcattctgactggtggcatagagttctatctgcagacgagcattgtcgggaaccagagtcatgaccggttcagatgccctgacagactgtccctgtctgatcagcacggcagcaacggttccggataccggtgacgtcagcgtaaagttctcctgtccggccagttcgtcctgctgttgcctgatcccctgcaactgcctgtccagctcagctttacggctttccccctgaacgatgagatggttaagatcttcttcagcagcctccattgcagtgtgtagttgcagaagcccctgacgctgatcctcaacgttttgcagtgacgtagaaacgtcaatttgcttctgctggtattcgatatctgatacgtaatgcgttccagccagttttttatagcgctccatgacggacacggccagcgtggcctgatgcccggcaagcgacaggcgcagctctgcacttttaatctgtggtttcagggaggcaattcgctgtagtatggcctgctgctgctgactgttgtcacgcaattccagagtctgctgggaagacagcatggcatactgagtcttcagggacatgctcatggctgccagtgtacctgtgccctgcccgttgtaatgctcgccgctgatatggtaaacaggctcaccggcagctacgtgctgaccttctgaaacagtctgtcgggtgacgtatccggcatattgcggtgtgattttcaccagtcctgatgaaggcatcactatacctgtaagatgagccttgcgggtataactaccataataaacgaataatgagacgctcagaaatataagtaatgtagcaacggcgcatactgacatgccaaatgacgcgggtaagacaatatctccgtactctgtatcactgttatgttctaatgcttcctccctgaatatactcatggttataaaatattttgaatatttaataatataacaaaggtattcatgcctgaatgtattaacataggataaaaaagggatctgcttttaattctgacccctaataacagcatagatacaacaaaaaggactgattgatccagaacattataatactgaaagtgcatcagagagaaaaacaaagatgttatcacacagggaatgataatgttttccttgaaaattgaacgcagaaatccaaaggcacaggctctgtatacgatctcctcatagtagggtacgaccagtgtcatcactaaaactgttatccagtttatctgtgatggttcgtaatgatataaatagtctctgtagcagtaaacagctaactgtatcagaataattataaggaatgacagggctgaaaatttaactgtagctgagtctgttcttatttttattctacactcaggaaattcctttaggtaaaataagtaaacaagagttgaaattagaatctctgtaatgaacacaatgaaaaatgcaatatccagtccaacataacgcagtgtaaaggttggcgataatgttacaatagttgatataataaccactgcaagaaaaataatagcgctgtatttactatatctaaactggctcgaacgttcatccattacaaaacatcactttatgaaggttattatttttgctaaaactatatctgtaaatttagcagtggaaaacaaggtgatcaatgtaattactatttttgaaaattcctcaggcaacgaaaaccatgacaaatcaataatggaggaaccacttaatagcgcaaaacccagtaataaacttaatataaaagacaataatttaataaacactgcagtatcctccgttgatacttacttatcgactacaggtaccaccgacgttacttcctgaacaattactggaaccagctctgtttccgcctccgttaccatttcctccaccggagagacactggccgataactgccccaccaatagttcctctggtcattcctacaggaccacctttaatggcaccaccaaccattccactaaagacagcattagcacatttaactgtgctcgggtcactgtaaatgtgtgtcggtgcatttcgtcccagtgagttacgggctccggtgttacgactacgacttccgcctccttcataattactgtttgcgttgccaccactgactaatgcaatttcatcaaaactcaattctctgatatttgacat

SEQ ID NO: 2 - DNA, 363 bp, Escherichia coli
atgtcaaatatcagagaattgagttttgatgaaattgcattagtcagtggtggcaacgcaaacagtaattatgaaggaggcggaagtcgtagtcgtaacaccggagcccgtaactcactgggacgaaatgcaccgacacacatttacagtgacccgagcacagttaaatgtgctaatgctgtctttagtggaatggttggtggtgccattaaaggtggtcctgtaggaatgaccagaggaactattggtggggcagttatcggccagtgtctctccggtggaggaaatggtaacggaggcggaaacagagctggttccagtaattgttcaggaagtaacgtcggtggtacctgtagtcgataa

SEQ ID NO: 3 - DNA, 651 bp, Escherichia coli
atggatgaacgttcgagccagtttagatatagtaaatacagcgctattatttttcttgcagtggttattatatcaactattgtaacattatcgccaacctttacactgcgttatgttggactggatattgcatttttcattgtgttcattacagagattctaatttcaactcttgtttacttattttacctaaaggaatttcctgagtgtagaataaaaataagaacagactcagctacagttaaattttcagccctgtcattccttataattattctgatacagttagctgtttactgctacagagactatttatatcattacgaaccatcacagataaactggataacagttttagtgatgacactggtcgtaccctactatgaggagatcgtatacagagcctgtgcctttggatttctgcgttcaattttcaaggaaaacattatcattccctgtgtgataacatctttgtttttctctctgatgcactttcagtattataatgttctggatcaatcagtcctttttgttgtatctatgctgttattaggggtcagaattaaaagcagatcccttttttatcctatgttaatacattcaggcatgaatacctttgttatattattaaatattcaaaatattttataa

SEQ ID NO: 4 - DNA, 1254 bp, Escherichia coli
atgagtatattcagggaggaagcattagaacataacagtgatacagagtacggagatattgtcttacccgcgtcatttggcatgtcagtatgcgccgttgctacattacttatatttctgagcgtctcattattcgtttattatggtagttatacccgcaaggctcatcttacaggtatagtgatgccttcatcaggactggtgaaaatcacaccgcaatatgccggatacgtcacccgacagactgtttcagaaggtcagcacgtagctgccggtgagcctgtttaccatatcagcggcgagcattacaacgggcagggcacaggtacactggcagccatgagcatgtccctgaagactcagtatgccatgctgtcttcccagcagactctggaattgcgtgacaacagtcagcagcagcaggccatactacagcgaattgcctccctgaaaccacagattaaaagtgcagagctgcgcctgtcgcttgccgggcatcaggccacgctggccgtgtccgtcatggagcgctataaaaaactggctggaacgcattacgtatcagatatcgaataccagcagaagcaaattgacgtttctacgtcactgcaaaacgttgaggatcagcgtcaggggcttctgcaactacacactgcaatggaggctgctgaagaagatcttaaccatctcatcgttcagggggaaagccgtaaagctgagctggacaggcagttgcaggggatcaggcaacagcaggacgaactggccggacaggagaactttacgctgacgtcaccggtatccggaaccgttgctgccgtgctgatcagacagggacagtctgtcagggcatctgaaccggtcatgactctggttcccgacaatgctcgtctgcagatagaactctatgccaccagtcagaatgccggttttattcagccggggcagcgtgttgcacttcggttcgccgcattcccctatcagaagtttggcgttcagtacgggactatccgagagataagccgtacaacgctgaccccttcggatttactctccgtatcccctgtgacctggaaagagaatgaagggcactaccgcgtcatcgtcgaacctgagaataccttcattctggcttatggaaaacaggaacccctgcgccccggcatgacccttgagggggatgtcagcctggatacccgccatttgtgggagtggctgaccgagccactctggagcctgaaaggaaaactgtaa

SEQ ID NO: 5 - DNA, 2178 bp, Escherichia coli
atggatacccttaaatggaccgggagaaaacagctccccctcatccggcagacagagtctgcggaatgtggtctggcatgtctggtcatgatggcctgctggcatggtctgcagacagatttacccaccctgcgcgaacgtttcagtatcagcacacaggggatgaccctgcagaggctgatagagtgtgcagccggtatccggttgtcctcccgggccgtccggctggagccggaagacctgaaatccctttctctcccgtgcattctccactggaacatgaaccatttcgtcgtgctttataaagttcgcgggagcaggctggttatccacgacccggacaaagggaagataacgctctccctgcaggatgcaggaaaacatttcaccggtgtcgcgctggagttgacgccggccagtgattttaccgcccgggatgaaagaaaaaaaattcgcctgcgccaactgacaggcaggacacccgggctgctggccgcgatgtcgcgcatcatcatttttgccctggcgctggaaatcctgacgctgagtagtccacttctcaaccagctggtaattgatgaagtgctggttgccgccgatcgaagtctcctgaccgttataatcattgccctgctgctgctgtcaatgacccagatgctgctgtcgctggcacgccagtgggccagtataactctgtccgtcaattttaacatgcagtggacagcccgggttttccatcatctggtgcgtctcccgctgggctggtttgatgcgcgcagtaagggaagtatcaatgcccgttttgaggccgtggataccatccaggaggcgctgaccacgcaggtactggaaggtattctggacgtgttgctcgtgataactgcgctctgtatgatgctgctgtacagtccggaaatgacgctgatagccgtactggcagccattatctacggtgtgctgagggcgttgtggtatccgtcccttcgacagtcggcagaagatgcctgggacgcaggtgccagggagtccgggcattttcttgaaaccctcaacggtattctgagcctgagaattaacggcgtgacagcgcaccgtgaggccgcctggctgaacctcaacattgttcgcaggaatacgcagctgcgacagagccgtcagctgatgtgctatgacattgcacacaccctgaccggcagcgtggtatcagcgattattttgtggaaaggtgccggggaagtactgaacggtaccttcaccgtcggcatgctggttgcttatttgtcctaccagatgcgtttttcatccagtatcagcagcctgaccgataagtatttttcctggcgtatgcttgatatctacaacgaacgcctggctgacattgtgctgaccccgacagaagactatcagcaacaacccgctcaggaagacaatgatacttctgtcttcccgtccgtattccggggggatgtggccgacggtgcccgtgttcctttatcccttgagcatataaccttcagccacaagggagggaataaaccgatactccgcggggtgtcgctgacactccatcctggcgaggtgatggccattaccggtcaatccggatgcgggaaatcgacactggtgaagcttatcctggggatccacatcccggatgagggaacaatcagggcatttggcataccgcatacccacccggattattttcaggtacgtcagcgaatcggtaccgtactgcaggatgaccatcttttcaggggctccattgcagataacatcatttttttcagtggagaccgcaatcatgaacgaatgatccagtgcgccagactggcacttatcgacagcgatatcatgtctatgccgatggggtatcagacgctgattggtgaaaccggtggagggctttccggagggcaaaaacaacgcattctgctggccagggctttgtacaagaaaccgggatttttattactggatgaagcgaccagccatcttgatgttgaaagtgaaattcagataagccagacactacgtcagctggggcttcctgtcctgctgatagcccatcgcccggaaaccatagcctctgcagaccgggttctgtatctggcagacggctgctttatggagctgaggcacaagcgcaccattgatgatgggctgaacaaaaattaa

SEQ ID NO: 6 - protein, 120 aa, Escherichia coli
MSNIRELSFDEIALVSGGNANSNYEGGGSRSRNTGARNSLGRNAPTHIYSDPSTVKCANAVFSGMVGGAIKGGPVGMTRGTIGGAVIGQCLSGGGNGNGGGNRAGSSNCSGSNVGGTCSR

SEQ ID NO: 7 - protein, 102 aa, Escherichia coli
NANSNYEGGGSRSRNTGARNSLGRNAPTHIYSDPSTVKCANAVFSGMVGGAIKGGPVGMTRGTIGGAVIGQCLSGGGNGNGGGNRAGSSNCSGSNVGGTCSR

SEQ ID NO: 8 - DNA, 19 bp, Artificial Sequence
  pSYM1 sequencing primer
cagctggatatcctgcgcg

SEQ ID NO: 9 - DNA, 19 bp, Artificial Sequence
  pSYM1 sequencing primer
ggttgcccggcatccaacg

SEQ ID NO: 10 - DNA, 31 bp, Artificial Sequence
  pAZ6/pAZ8 cloning primer
tcaattgtgtcgactcaattactcttgtgag

SEQ ID NO: 11 - DNA, 34 bp, Artificial Sequence
  pAZ6 cloning primer
catgtaatagtgctagcatgttaaaatttataag

SEQ ID NO: 12 - DNA, 35 bp, Artificial Sequence
  pAZ8, pAZ12 cloning primer
caaaaataatagctagcaagtgatgttttgtaatg

SEQ ID NO: 13 - DNA, 28 bp, Artificial Sequence
  pAZ9 cloning primer
ctcgaattcatccattacaaaacatcac

SEQ ID NO: 14 - DNA, 30 bp, Artificial Sequence
  pAZ9 cloning primer
ctggctgcagtaattgttcaggaagtaacg

SEQ ID NO: 15 - DNA, 27 bp, Artificial Sequence
  pAZ10 cloning primer
taggaattcagaggaactattggtggg

SEQ ID NO: 16 - DNA, 37 bp, Artificial Sequence
  pAZ10 cloning primer
ctccgctgcagacttacttatcgactacaggtaccac

SEQ ID NO: 17 - DNA, 37 bp, Artificial Sequence
  pAZ11 cloning primer
gttagaattcataagagggatttttatgtcaaatatc

SEQ ID NO: 18 - DNA, 35 bp, Artificial Sequence
  pAZ11 cloning primer
gttgatactgcagcttatcgactacaggtaccacc

SEQ ID NO: 19 - DNA, 31 bp, Artificial Sequence
  pAZ12 cloning primer
cccaagcttagttaaatgtgctaatgctgtc

SEQ ID NO: 20 - DNA, 18 bp, Artificial Sequence
  pAZ12 cloning primer
ggcatcggtcgacgcaac

SEQ ID NO: 21 - DNA, 32 bp, Artificial Sequence
  pAZ14 cloning primer
cattgctagccatcacagataaactggataac

SEQ ID NO: 22 - DNA, 33 bp, Artificial Sequence
  pAZ13, pAZ14 cloning primer
ccctgagtcgactcatggttataaaatattttg

SEQ ID NO: 23 - DNA, 22 bp, Artificial Sequence
  inhibition control
atggctatcgacgaaaacaaac

SEQ ID NO: 24 - DNA, 24 bp, Artificial Sequence
  inhibition control
ttaaaaatcttcgttagtttctgc

SEQ ID NO: 25 - DNA, 23 bp, Artificial Sequence
  mcsS screening primer
atgtcaaatatcagagaattgag

SEQ ID NO: 26 - DNA, 21 bp, Artificial Sequence
  mcsS screening primer
ttatcgactacaggtaccacc